(19) United States
(12) Patent Application Publication
(10) Pub. No.: US 2006/0031026 A1
(43) Pub. Date: Feb. 9, 2006

(54) Method and system for extracting and visualizing secondary RNA structure elements from protein-RNA complexes

(75) Inventors: Kyung Sook Han, Yeonsu-gu (KR); Daeho Lim, Seoul (KR)

Correspondence Address: BACHMAN & LAPOINTE, P.C., 900 CHAPEL STREET, SUITE 1201, NEW HAVEN, CT, 06510, US

(73) Assignee: Inha-Industry Partnership

(21) Appl. No.: 11/146,349

(22) Filed: Jun. 6, 2005

(30) Foreign Application Priority Data
Aug. 9, 2004 (KR) KR2004-0062552

(51) Int. Cl. G06F 19/00; G01N 33/48; G01N 33/50

(52) U.S. Cl. 702/20

(57) ABSTRACT

The present invention relates to a method for extracting and visualizing RNA structure elements comprising extracting secondary and tertiary RNA structural elements by applying data mining technique to three-dimensional atomic coordinate data of RNA obtained from protein data bank (PDB) and visualizing a general structure of RNA and the bond between nucleic acids forming a RNA molecule, based on the information on said extracted structural elements and a system for performing said method. The system of the present invention comprises the first means for extracting structural elements of a RNA molecule from a database and the second mean for visualizing a structure of the RNA molecule. The system of the present invention will be a great aid for the prediction of a structure of RNA or a bond of a protein-RNA complex because it uses the data kept in protein data bank (PDB) as input data and provides the exact data of structural elements of RNA molecule and a concretely visualized structure.

### FIELD OF THE INVENTION

The present invention relates to a method for extracting and visualizing RNA structure elements comprising extracting secondary and tertiary RNA structural elements by applying data mining technique to three-dimensional atomic coordinate data of RNA obtained from protein data bank (PDB) and visualizing a general structure of RNA and the bond between nucleic acids forming a RNA molecule, based on the information on said extracted structural elements and a system for performing said method.

### BACKGROUND

In general, bioinformatics is interpreted as a combination of bioscience and information science. Prior art for bioinformatics are as follows.

Genomic base sequence analysis technique: Japanese Patent Publication H05-168500 [Method for determining nucleic acid sequence]

Gene information analysis technique: Japanese Patent Publication H10-045795 [Protein database system and estimating method for protein structure and functional region]

Hyperstructure analysis technique: Japanese Patent Publication H09-159666 [Prediction method and apparatus for the secondary structure of protein]

Network identifying and simulation technique: Japanese Patent Publication 2001-0005797 [Network estimating method and apparatus]

In this field of bioinformatics, extraction of structural elements of a RNA molecule has been performed by manual operation. Therefore, an automatic system is required to extract secondary and tertiary structural elements of RNA and to visualize an actual structure of RNA based on said extracted structural information.

### SUMMARY OF THE INVENTION

It is an object of the present invention to provide a method for extracting and visualizing secondary and tertiary structure of RNA and an automatic system for performing said method.

### DETAILED DESCRIPTION OF PREFERRED EMBODIMENTS

In order to achieve the above-mentioned object, the present invention provide a system for extracting and visualizing secondary and tertiary structure of RNA from protein-RNA complexes, comprising the first means for extracting structural elements of a RNA molecule from a database and the second means for visualizing a structure of the RNA molecule.

In an embodiment of the system of the present invention, the first means is executed by an algorithm for extracting secondary and tertiary structural elements of RNA molecule from a database and the second means is executed by an algorithm for visualizing a secondary and tertiary structure of RNA based on the data of structural elements of RNA molecule extracted from the first means and an output device. In a preferred embodiment of the system of the present invention, the first means comprises a module for extracting the data of hydrogen bond; a module for classifying the data of hydrogen bonds forming base pairs into one of 28 types; a module for extracting the data of nucleic acid sequences of RNA; a module for extracting the data of structural elements of RNA. In a more preferred embodiment of the system of the present invention, the module for extracting data of hydrogen bond comprises an algorithm for extracting the data of nucleotides and hydrogen bonds thereof forming a RNA molecule from the data of atomic coordinates of a RNA or a protein-RNA complex molecule obtained from a database, one for selecting the data of hydrogen bonds generated especially by bases of nucleotides among said data of hydrogen bond, and one for extracting only those data of hydrogen bond forming base pair by processing said selected data of hydrogen bonds. In another preferred embodiment of the system of the present invention, the module for extracting the data of structural elements is executed by integrating the data of hydrogen bonds generating the classified bas pairs and the data of nucleic acid sequence of RNA and processing thereof. In a preferred embodiment of the system of the present invention, the database is protein data bank (PDB) but not limited to it. In a more preferred embodiment of the system of the present invention, the data of structural elements of RNA molecule is one of atomic coordinates of RNA or a protein-RNA complex kept in PDB file.

In another embodiment of the system of the present invention, the second means comprises a module for extracting the data of structural elements of RNA; a module for generating the data for visualization; and a module for visualizing the structure of RNA based on the data for visualization. In a more preferred embodiment of the system of the present invention, the module for extracting the data of structural elements of RNA is executed by integrating the data of hydrogen bonds generating the classified base pairs and the data of nucleic acid sequence of RNA and processing thereof. In another preferred embodiment of the system of the present invention, wherein the module for generating the data for visualization is executed by integrating the extracted data of nucleic acid coordinates and the data of structural elements of RNA extracted by the module for extracting of structural elements of RNA. In a more preferred embodiment of the system of the present invention, the module for visualizing the structure of RNA comprises an algorithm for visualizing structure of RNA based on the data for visualization generated by the module for generating of the data for visualization and an output device.

In a preferred embodiment of the system of the present invention, the output device is a monitor comprising CRT, LCD, PDP, OLED or LED, a printer, a plotter or a non-volatile memory comprising a flash memory such as SD(SanDisk), CF-memory(Compact Flash memory), SMC(Smart Media Card) and Memory Stick, a harddisk drive, a floppy diskette, an opictical disk such as CD-R, CD-RW, MD, DVD-R, DVD-RW, DVD±RW, DVD+RW and DVD-RAM. In such non-volatile memory, the data for visualization can be recorded as a graphic format file comprising JPG, TIF, PDF, GIF, WMF or TGA.

In the most preferred embodiment of the present invention, the system for extracting and visualizing secondary and tertiary structure of RNA from protein-RNA complexes, comprises a module for extracting the data of hydrogen bond, which includes an algorithm for extracting the data of nucleotides and hydrogen bonds thereof forming a RNA molecule from the data of atomic coordinates of a RNA or a protein-RNA complex molecule obtained from PDB (protein data bank), one for selecting the data of hydrogen bonds generated especially by bases of nucleotides among said data of hydrogen bond, and one for extracting only those data of hydrogen bond forming base pair by processing said selected data of hydrogen bonds; a module for classifying the data of hydrogen bonds forming base pairs into one of 28 types; a module for extracting the data of nucleic acid sequences of RNA; a module for extracting the data of structural elements of RNA, which extracts structural elements of RNA molecule by integrating the data of hydrogen bonds generating the classified base pairs and the data of nucleic acid sequence of RNA and processing thereof; a module for extracting the data of nucleic acid coordinates, which classifies the data of three-dimensional atomic coordinates kept in PDB according to types of nucleic acid and extracts the data of nucleic acid coordinates by calculating the mean value of said data of three-dimensional atomic coordinates; a module for generating the data for visualization by integrating the extracted data of nucleic acid coordinates and the data of structural elements of RNA extracted by the module for extracting of structural elements of RNA; and a module for visualizing the structure of RNA based on the data for visualization.

In order to achieve the above-mentioned object, the present invention provides a method for extracting and visualizing secondary RNA structure from protein-RNA complexes using said system, comprising the first step of extracting data of secondary and tertiary structural elements of RNA from a database; and the second step of visualizing a whole structure of RNA based on said extracted data of structural elements.

In a preferred embodiment of the method of the present invention, the first step comprises the following steps:
i) extracting the data of hydrogen bond;
ii) classifying the data of hydrogen bonds forming base pairs into one of 28 types;
iii) extracting the data of nucleic acid sequences of RNA; and
iv) extracting the data of structural elements of RNA.

In a more preferred embodiment of the method, the step i) comprises extracting the data of nucleotides and hydrogen bonds thereof forming a RNA molecule from the data of atomic coordinates of a RNA or a protein-RNA complex molecule obtained from PDB, selecting the data of hydrogen bonds generated especially by bases of nucleotides among said data of hydrogen bond, and extracting only those data of hydrogen bond forming base pair by processing said selected data of hydrogen bonds and the step iv) is executed by integrating the data of hydrogen bonds generating the classified base pairs and the data of nucleic acid sequence of RNA and processing thereof.

In another preferred embodiment of the method according to 15, wherein the second step comprises the following steps:
i) extracting the data of nucleic acid coordinates;
ii) generating the data for visualization; and
iii) visualizing the structure of RNA based on the data for visualization on the output device of said system.

In a more preferred embodiment of the method, the step i) is executed by classifying the data of three-dimensional atomic coordinates kept in PDB according to types of nucleic acid and extracts the data of nucleic acid coordinates by calculating the mean value of said data of three-dimensional atomic coordinates and step ii) executed by integrating the extracted data of nucleic acid coordinates and the data of structural elements of RNA.

In the most preferred embodiment of the present invention, the method for extracting and visualizing secondary RNA structure from protein-RNA complexes comprising the following steps:
i) extracting the data of hydrogen bond, which comprises extracting the data of nucleotides and hydrogen bonds thereof forming a RNA molecule from the data of atomic coordinates of a RNA or a protein-RNA complex molecule obtained from PDB, selecting the data of hydrogen bonds generated especially by bases of nucleotides among said data of hydrogen bond, and extracting only those data of hydrogen bond forming base pair by processing said selected data of hydrogen bonds;
ii) classifying the data of hydrogen bonds forming base pairs into one of 28 types;
iii) extracting the data of nucleic acid sequences of RNA;
iv) extracting the data of structural elements of RNA, which extracts structural elements of RNA molecule by integrating the data of hydrogen bonds generating the classified base pairs and the data of nucleic acid sequence of RNA and processing thereof;
v) extracting the data of nucleic acid coordinates, which classifies the data of three-dimensional atomic coordinates kept in PDB according to types of nucleic acid and extracts the data of nucleic acid coordinates by calculating the mean value of the data of three-dimensional atomic coordinates;
vii) generating the data for visualization by integrating the extracted data of nucleic acid coordinates and the data of structural elements of RNA extracted by the module for extracting of structural elements of RNA; and
viii) visualizing the structure of RNA based on the data for visualization on the output device of said system.

### BRIEF DESCRIPTION OF THE DRAWINGS

The application of the preferred embodiments of the present invention is best understood with reference to the accompanying drawings, wherein:

FIG. 1 is a set of schematic diagrams showing the 4 most representative base pairs among 28 types of base pairs.

FIG. 2 is a schematic diagram showing a system for extracting and visualizing secondary and tertiary structure of RNA of the present invention.

FIG. 3 is a flow chart showing the processes of visualization carried out by the system shown in FIG. 2.

FIG. 4 is a table showing the information on tertiary structural elements of mouse mammary tumor virus (PDB ID: 1RNK) obtained by the system of the present invention.

FIG. 5 is a schematic diagram visualizing the structure of a RNA molecule, using the system of the present invention, based on the data of structural elements.

FIG. 6 is a schematic diagram visualizing the structure of a RNA molecule (PDB ID: 1DFU) having two chains, using the system of the present invention including algorithms for extracting of structural elements and visualizing thereof.

FIG. 7 is a schematic diagram visualizing the structure of tRNA (PDB ID: 1EHZ), one of types of RNA molecules, using the system of the present invention including algorithms for extracting of structural elements and visualizing thereof.

FIG. 8 is a schematic diagram visualizing the structure of a RNA molecule (PDB ID: 1FG0) having a complicated structure, using the system of the present invention including algorithms for extracting of structural elements and visualizing thereof.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

In the statement of the present invention, terms are defined as follows.

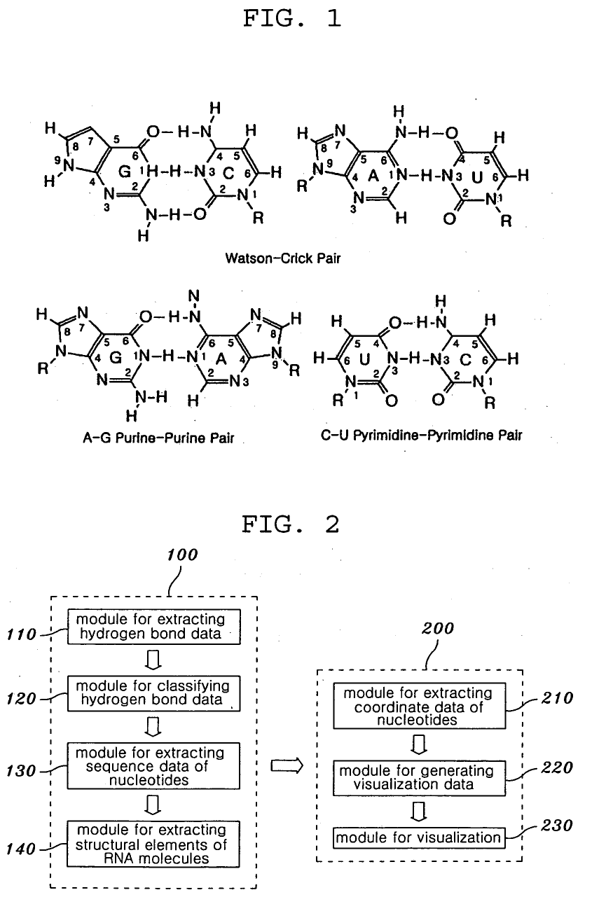

Base pair: RNA consists of nucleic acid molecules and each nucleic acid consists of base, phosphate and sugar. A base pair is formed when one base is paired with another base by stable hydrogen bonds. Base pairs are classified into canonical base pairs and non-canonical base pairs according to types of nucleic acid and hydrogen bonds. More particularly, they are classified into 28 types. FIG. 1 shows the most representative four base pairs among 28 types of base pairs.

Base pairing rule: Atoms costituting base of nucleic acid have fixed numbers (see FIG. 1). These fixed numbers of atoms provide a very important clue for distinguishing a base pair. For example, G-C Watson-Crick pair is formed by hydrogen bond between No. 6 oxygen, No. 1 hydrogen and No. 2 nitrogen of guanine and No. 4 nitrogen, No. 3 nitrogen and No. 2 oxygen of cytosine. A-U Watson-Crick pair also has two hydrogen bonds between atoms with specific numbers. Base pairs are classified into 28 types including Wooble pair, Pyrimidine-Pyrimidine pair and Purine-Purine pair in addition to Watson-Crick pair. Base pairs are formed and classified by such hydrogen bonds between atoms with fixed numbers, and this is called as base pairing rule. This rule plays an important role in extracting hydrogen bonds forming base pairs with algorithm for extracting structural elements of RNA and in classifying hydrogen bonds based on 28 types of base pairs.

RNA structure: RNA structure is stably formed by hydrogen bonds between nucleic acids consisting of a RNA molecule. Thus, the data of structural elements of a RNA molecule provides the information on bonds between nucleic acids necessary for constructing a stable molecular structure of RNA.

PDB (protein data bank): PDB is a database and format of files, which describe the 3D structure of a protein or nucleic acid, as determined by X-ray crystallography or nuclear magnetic resonance (NMR) imaging. The molecules described by the files are usually viewed locally by dedicated software, or can be visualized on the World Wide Web (http://www.rcsb.org/pdb).

Output device: Output device is any peripheral that receives output from a computer. The examples are monitors, plotters, floppy diskettes, hard disk drives and optical disks such as CD-R, CD-RW, DVD±RW, DVD+RW, DVD-RW, DVD-RAM and MD. The monitor (or visual display unit) as a typical output device displays text and graphics.

### Example 1

### Extraction of Structural Elements of RNA and Visualizing System

FIG. 2 is a schematic diagram showing a system for extracting and visualizing secondary and tertiary structure of RNA of the present invention.

As shown in FIG. 2, the system comprises two means. The first means is so-called an extraction tool (100) for extracting structural elements of a RNA molecule, executed by an algorithm for extracting secondary and tertiary structural elements of a RNA molecule, and the second means is a visualization tool (200) for visualizing the structure or molecules of RNA, executed by an algorithm for visualizing a secondary and tertiary structure of RNA based on the data of structural elements of RNA molecule extracted from the first means and an output device.

The first means for extracting structural elements (100) comprises a module for extracting the data of hydrogen bonds (110), a module for classifying the data of the above hydrogen bonds (120), a module for extracting the data of nucleic acid sequences (130) and a module for extracting the data of structural elements of a RNA molecule.

The module for extracting the data of hydrogen bonds (110) extracts the data of hydrogen bonds and the data of nucleic acids forming a RNA molecule from the data of atomic coordinates of RNA or a protein-RNA complex kept in PDB. Among hydrogen bonds, specific hydrogen bonds generated between bases are extracted and processed to extract the data of hydrogen bonds generating base pairs. The module for classifying the data of hydrogen bonds (120) classifies the data of hydrogen bonds generating base pairs into 28 types of them. The module for extracting the data of structural elements of a RNA molecule (140) executes an extracting process by integrating the data of hydrogen bonds forming the classified base pairs and the data of nucleic acid sequence of RNA extracted by the module for extracting sequence data of nucleotides (130) and processing thereof, and then provides the data of structural elements of a RNA molecule.

The second means of the system, visualizing the structure of RNA molecule (200) based on the data of structural elements extracted by the first means, comprises a module for extracting the data of nucleic acid coordinates (210), a module for generating visualizing data (220) and a module for final visualization (230).

The module for extracting the data of nucleic acid coordinates (210) classifies three-dimensional atomic coordinates data kept in PDB into one of nucleic acid types and calculates the mean value of the three-dimensional atomic coordinates to extract the data of nucleic acid coordinates. The module for generating visualizing data (230) produces the visualizing data by integrating the obtained data of nucleic acid coordinates and the data of structural elements of RNA molecule obtained from the first means of the system. The module for visualization (230) executes a visualization process using the visualizing data generated by the module for generating visualizing data and an output device.

### Example 2

### Extraction of Structural Elements of RNA and Visualizing Algorithm

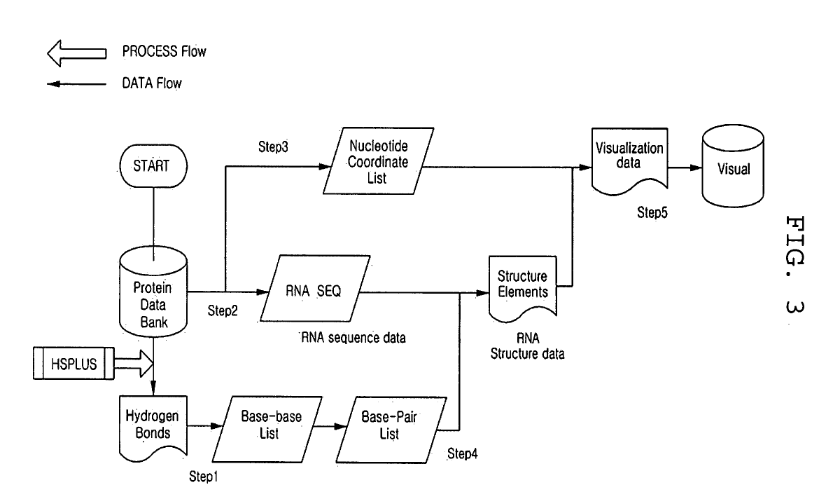

FIG. 3 is a flow chart showing the processes of visualization carried out by the system shown in FIG. 2.

In FIG. 3, step 1, step 2 and step 3 represent the first algorithm executing extraction of secondary or tertiary structural elements of RNA from PDB data, and step 4 and step 5 represent the second algorithm executing visualization based on the data of structural elements of the molecule obtained from the first algorithm.

The present invention is more particularly described hereinafter with reference to FIG. 2 and FIG. 3.

Step 1: Firstly, with the module for extracting the data of hydrogen bonds (110), the data of hydrogen bonds between atoms extracted from PDB file are analyzed by HBPLUS application, resulting in selection of hydrogen bonds generated between bases of nucleic acid. The data of hydrogen bonds obtained by HBPLUS application means the data of hydrogen bonds generated among all the atoms constituting a molecule. Thus, it is required to extract such hydrogen bonds as generated only between bases, in order to obtain the data of hydrogen bonds involved in base pairs. That is, hydrogen bonds between bases are extracted firstly and then among them, only the hydrogen bonds generating base pairs are extracted. In the system of the present invention, an algorithm enabling the extraction of structural elements of RNA molecule accepts only the base pairs having more than 2 hydrogen bonds between bases. Therefore, even though it is a hydrogen bond between bases, it will be excluded if it does not generate base pair.

After obtaining hydrogen bonds generating base pairs, those bonds are classified into one of 28 types, with the hydrogen bond classifying module (120). At this time, the above-mentioned base-pairing rule is applied to distinguish those hydrogen bonds generating base pairs, which will be used as a standard for base pair classification.

Step 2: It is important to gather information on nucleic acid constituting RNA in order to extract the data of secondary and tertiary structural elements of RNA. PDB file includes the data of nucleic acid constituting RNA at the atomic level. Therefore, in this step, the data of nucleic acid sequence were extracted by classifying the data of atoms constituting RNA obtained from PDB file into each unit of nucleic acid, with the module for extracting the data of nucleic acid sequences (130). The data of nucleic acid sequences provide a huge amount of information on nucleic acid constituting RNA.

Step 3: This step is to extract the data of structural elements of RNA with the module for extracting structural elements of RNA (140). The data of hydrogen bonds involved in base pairs extracted in said step 1 and the data of nucleic acid sequences constructing RNA extracted in said step 2 are integrated to give the information on structural elements of RNA. Since the data of nucleic acid sequences obtained in the step 2 contain all the information on every nucleic acid constituting RNA, the bonds between one nucleic acid and another can be clearly explained by comparing the data of base pairs with the data of nucleic acid sequences, through which specific nucleic acids involved in constructing a stable structure through base pairs can be distinguished. Further, such information on nucleic acid bonds and base pairs can give a clue for understanding structural elements of a whole structure of RNA molecule.

Step 4: In this step, the data of nucleic acid coordinates constituting RNA are obtained through searching in PDB file with the module for extracting the data of nucleic acid coordinates (210). PDB file contains the data of all the atomic coordinates but the data of nucleic acid coordinates. Thus, in order to obtain the data of nucleic acid coordinates, an algorithm for visualization has to be executed, which defines an average atomic coordinate data of nucleic acid as nucleic acid coordinate data. In conclusion, the data of atomic coordinates constituting RNA are classified into one of nucleic acid types and a mean value of the data of atomic coordinates classified according to types of nucleic acids is calculated, resulting in the data of nucleic acid coordinates constituting RNA.

Step 5: This is the step for final visualization of the structure of RNA. The above-mentioned module 220 is used to integrate the data of structural elements of RNA extracted in said step 3 and the data of nucleic acid coordinates obtained in said step 4, resulting in extraction of visualizing data. Visualization of a whole structure of RNA molecule is finally accomplished with the visualizing module (230) based on the data for visualization.

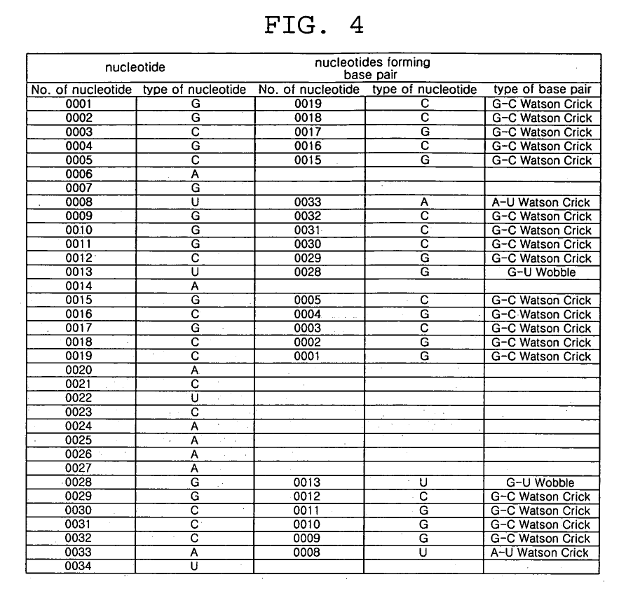

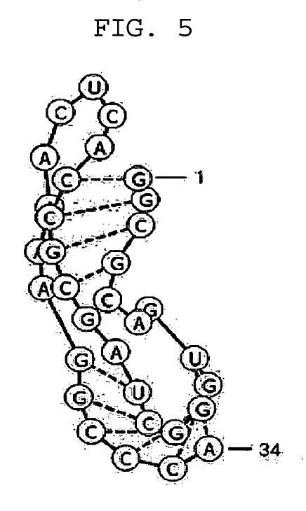

FIG. 4 is a table showing the information on tertiary structural elements of mouse mammary tumor virus (PDB ID: 1RNK) obtained by the system of the present invention. The first and the second column in the table represent all the types and fixed numbers of nucleic acid in RNA, and the third and the fourth column represent nucleic acids base-pairing with each ones represented by the first two columns, respectively. The last column shows the types of base pairs generated by the two nucleic acids. The table in FIG. 4 shows the types of nucleic acids constituting mouse mammary tumor virus RNA and bonds between one nucleic acid and another. FIG. 5 is a schematic diagram visualizing the structure of a RNA molecule, using the system of the present invention, based on the data of structural elements. Each node represents nucleic acid constituting RNA, and the solid blue line represents a phosphodiester backbone linking nucleic acids forming a chain of RNA molecule. Red dotted lines represent base pairs generated by hydrogen bonds between nucleic acids resulting in a stable structure of RNA molecule.

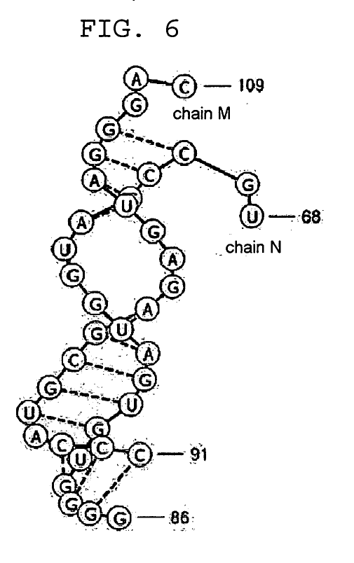

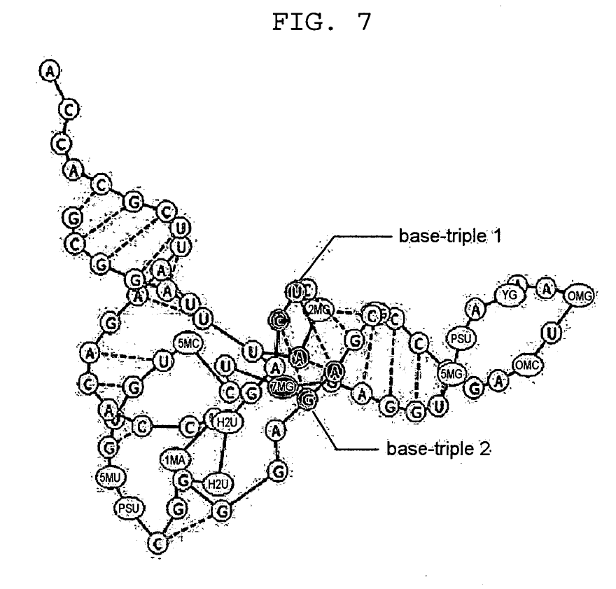

FIG. 6 is a schematic diagram showing the visualization of the structure of a RNA molecule (PDB ID: 1DFU) having two chains, using extracting and visualizing algorithm of the system of the present invention. 1DFU RNA molecule has two chains, which are M chain and N chain. Owing to the base pairs generated between nucleic acids constituting each chain, the structure of RNA becomes stable. The algorithm of the system of the present invention is to extract all the data of base pairs formed between nucleic acids, based on the data of hydrogen bonds between atoms constituting RNA molecule. Thus, it enables extraction of not only the data of base pairs in an identical chain but also the data of base pairs generated between heterogeneous chains.

Said FIG. 6 shows a RNA molecule having a stable structure generated by base-pairing between heterogeneous chains clearly. Base-triple structure playing an important role in establishing a stable tertiary structure of RNA molecule is generated when one of the two bases formed a base pair together is linked to another base again to make another base pair. The system of the present invention facilitates searching the base-triple structure because it is designed to extract all the data of structural elements of RNA based on base pair formed between nucleic acids.

FIG. 7 is a schematic diagram visualizing the structure of tRNA (PDB ID: 1EHZ), one of types of RNA molecules, using the system of the present invention including algorithms for extracting of structural elements and visualizing thereof. Bases marked in blue and yellow color are those forming base-triple structure.

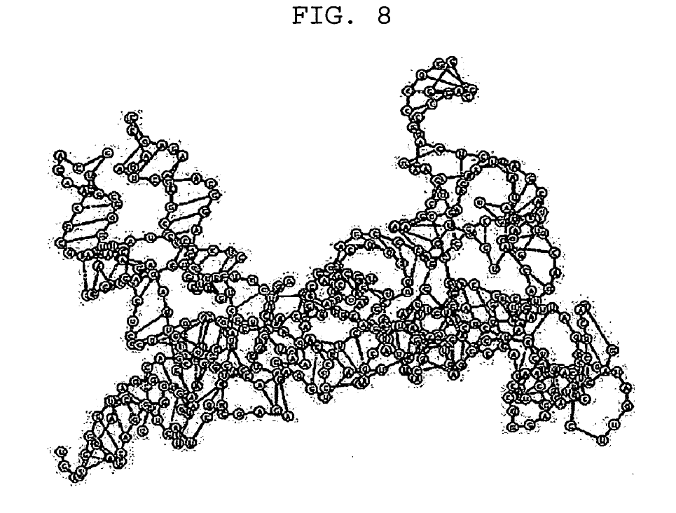

According to the system of the present invention comprising a means for extraction of secondary structural elements of RNA and one for visualizing thereof, structural elements of any RNA molecule can be extracted and visualized only if the data of three-dimensional atomic coordinates of the RNA is kept in PDB file. FIG. 8 is a schematic diagram visualizing the structure of a RNA molecule (PDB ID: 1FG0) having a complicated structure, using the system of the present invention including algorithms for extracting of structural elements and visualizing thereof.

### INDUSTRIAL APPLICABILITY

As explained hereinbefore, the present invention is the first attempt to visualize a structure of RNA by extracting secondary (28 base pairs) and tertiary structural elements (pseudoknot, base triple, etc) of RNA based on the data of three-dimensional atomic coordinates of RNA or a protein-RNA complex. The conventional manual operation for the extraction of structural elements of RNA can be substituted with an automatic method owing to the system of the present invention. In addition, the method of the invention will be a great aid for the prediction of a structure of RNA or a bond of a protein-RNA complex because it uses the data kept in protein data bank (PDB) as input data and provides the exact data of structural elements of RNA molecule and a concretely visualized structure.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A system for extracting and visualizing secondary and tertiary structure of RNA from protein-RNA complexes, comprising the first means for extracting structural elements of a RNA molecule from a database and the second means for visualizing a structure of the RNA molecule.

2. The system according to claim 1, wherein the first means is executed by an algorithm for extracting secondary and tertiary structural elements of RNA molecule from a database.

3. The system according to claim 1, the second means is executed by an algorithm for visualizing a secondary and tertiary structure of RNA based on the data of structural elements of RNA molecule extracted from the first means and an output device.

4. The system according to claim 1, wherein the first means comprises a module for extracting the data of hydrogen bond; a module for classifying the data of hydrogen bonds forming base pairs into one of 28 types; a module for extracting the data of nucleic acid sequences of RNA; a module for extracting the data of structural elements of RNA.

5. The system according to claim 4, wherein the module for extracting data of hydrogen bond comprises an algorithm for extracting the data of nucleotides and hydrogen bonds thereof forming a RNA molecule from the data of atomic coordinates of a RNA or a protein-RNA complex molecule obtained from a database, one for selecting the data of hydrogen bonds generated especially by bases of nucleotides among said data of hydrogen bond, and one for extracting only those data of hydrogen bond forming base pair by processing said selected data of hydrogen bonds.

6. The system according to claim 4, wherein the module for extracting the data of structural elements is executed by integrating the data of hydrogen bonds generating the classified bas pairs and the data of nucleic acid sequence of RNA and processing thereof.

7. The system according to claim 1, the database is protein data bank (PDB).

8. The system according to claim 1, wherein the data is one of atomic coordinates of RNA or a protein-RNA complex kept in PDB file.

9. The system according to claim 1, wherein the second means comprises a module for extracting the data of structural elements of RNA; a module for generating the data for visualization; and a module for visualizing the structure of RNA based on the data for visualization.

10. The system according to claim 9, wherein the module for extracting the data of structural elements of RNA is executed by integrating the data of hydrogen bonds generating the classified base pairs and the data of nucleic acid sequence of RNA and processing thereof.

11. The system according to claim 9, wherein the module for generating the data for visualization is executed by integrating the extracted data of nucleic acid coordinates and the data of structural elements of RNA extracted by the module for extracting of structural elements of RNA.

12. The system according to claim 9, wherein the module for visualizing the structure of RNA comprises an algorithm for visualizing structure of RNA based on the data for visualization generated by the module for generating of the data for visualization and an output device.

13. The system according to claim 3, the output device is a monitor comprising CRT, LCD, PDP, OLED or LED, a printer, a plotter or a non-volatile memory.

14. A system for extracting and visualizing secondary and tertiary structure of RNA from protein-RNA complexes, comprising a module for extracting the data of hydrogen bond, which includes an algorithm for extracting the data of nucleotides and hydrogen bonds thereof forming a RNA molecule from the data of atomic coordinates of a RNA or a protein-RNA complex molecule obtained from PDB (protein data bank), one for selecting the data of hydrogen bonds generated especially by bases of nucleotides among said data of hydrogen bond, and one for extracting only those data of hydrogen bond forming base pair by processing said selected data of hydrogen bonds; a module for classifying the data of hydrogen bonds forming base pairs into one of 28 types; a module for extracting the data of nucleic acid sequences of RNA; a module for extracting the data of structural elements of RNA, which extracts structural elements of RNA molecule by integrating the data of hydrogen bonds generating the classified base pairs and the data of nucleic acid sequence of RNA and processing thereof; a module for extracting the data of nucleic acid coordinates, which classifies the data of three-dimensional atomic coordinates kept in PDB according to types of nucleic acid and extracts the data of nucleic acid coordinates by calculating the mean value of said data of three-dimensional atomic coordinates; a module for generating the data for visualization by integrating the extracted data of nucleic acid coordinates and the data of structural elements of RNA extracted by the module for extracting of structural elements of RNA; and a module for visualizing the structure of RNA based on the data for visualization.

15. A method for extracting and visualizing secondary RNA structure from protein-RNA complexes using the system of claim 1, comprising the first step of extracting data of secondary and tertiary structural elements of RNA from a database; and the second step of visualizing a whole structure of RNA based on said extracted data of structural elements.

16. The method according to claim 15, wherein the first step comprises the following steps:
i) extracting the data of hydrogen bond;
ii) classifying the data of hydrogen bonds forming base pairs into one of 28 types;
iii) extracting the data of nucleic acid sequences of RNA; and
iv) extracting the data of structural elements of RNA.

17. The method according to claim 16, wherein the step i) comprises extracting the data of nucleotides and hydrogen bonds thereof forming a RNA molecule from the data of atomic coordinates of a RNA or a protein-RNA complex molecule obtained from PDB, selecting the data of hydrogen bonds generated especially by bases of nucleotides among said data of hydrogen bond, and extracting only those data of hydrogen bond forming base pair by processing said selected data of hydrogen bonds.

18. The method according to claim 16, wherein the step iv) is executed by integrating the data of hydrogen bonds generating the classified base pairs and the data of nucleic acid sequence of RNA and processing thereof.

19. The method according to claim 15, wherein the second step comprises the following steps:
i) extracting the data of nucleic acid coordinates;
ii) generating the data for visualization; and
iii) visualizing the structure of RNA based on the data for visualization on the output device.

20. The method according to claim 19, wherein the step i) executed by classifying the data of three-dimensional atomic coordinates kept in PDB according to types of nucleic acid and extracts the data of nucleic acid coordinates by calculating the mean value of said data of three-dimensional atomic coordinates.

21. The method according to claim 19, wherein the step ii) executed by integrating the extracted data of nucleic acid coordinates and the data of structural elements of RNA extracted by the step iv) of claim 16.

22. A method for extracting and visualizing secondary RNA structure from protein-RNA complexes comprising the following steps:
i) extracting the data of hydrogen bond, which comprises extracting the data of nucleotides and hydrogen bonds thereof forming a RNA molecule from the data of atomic coordinates of a RNA or a protein-RNA complex molecule obtained from PDB, selecting the data of hydrogen bonds generated especially by bases of nucleotides among said data of hydrogen bond, and extracting only those data of hydrogen bond forming base pair by processing said selected data of hydrogen bonds;
ii) classifying the data of hydrogen bonds forming base pairs into one of 28 types;
iii) extracting the data of nucleic acid sequences of RNA;
iv) extracting the data of structural elements of RNA, which extracts structural elements of RNA molecule by integrating the data of hydrogen bonds generating the classified base pairs and the data of nucleic acid sequence of RNA and processing thereof;
v) extracting the data of nucleic acid coordinates, which classifies the data of three-dimensional atomic coordinates kept in PDB according to types of nucleic acid and extracts the data of nucleic acid coordinates by calculating the mean value of the data of three-dimensional atomic coordinates;
vi) generating the data for visualization by integrating the extracted data of nucleic acid coordinates and the data of structural elements of RNA extracted by the module for extracting of structural elements of RNA; and
vii) visualizing the structure of RNA based on the data for visualization on the output device of the system of claim 1.
